(19) United States
(12) Patent Application Publication
(10) Pub. No.: US 2021/0298967 A1
(43) Pub. Date: Sep. 30, 2021

(54) FOLDED ABSORBENT ARTICLE

(71) Applicant: Essity Hygiene and Health Aktiebolag, Goteborg (SE)

(72) Inventors: Robert Albino, Goteborg (SE); Erik Jamsa, Goteborg (SE)

(21) Appl. No.: 17/252,796

(22) PCT Filed: Jun. 26, 2018
(86) PCT No.: PCT/SE2018/050684

§ 371 Date: Dec. 16, 2020

(51) Int. Cl. A61F 13/551; A61F 13/15; A61F 13/49

(52) U.S. Cl. CPC A61F 13/55115; A61F 2013/530481; A61F 13/49; A61F 13/15747

(57) ABSTRACT

A folded absorbent article includes absorbent core sandwiched between a liquid-permeable topsheet and a liquid-impermeable backsheet. The absorbent article defines, in an unfolded and planar state, a longitudinal center line and a transverse center line Moreover, the absorbent article has a flexure-resistance of less than 450.0 grams-force. Each transverse side of the absorbent article is folded along a respective longitudinal fold line, such that each of transversely opposed side edges are folded onto a topsheet surface thereby forming a firstly folded absorbent article. The firstly folded absorbent article is thereafter folded along a first transverse fold line located in the region of the transverse center line thereby forming a secondly folded absorbent article. Finally, the secondly folded absorbent article is folded along a second transverse fold line located approximately in the middle between the transverse center line and a waist edge thereby forming a thirdly folded absorbent article.

### CROSS-REFERENCE TO RELATED APPLICATION

This application is a national phase entry of, and claims priority to, International Application No. PCT/SE2018/050684, filed Jun. 26, 2018, with the same title as listed above. The above-mentioned patent application is incorporated herein by reference in its entirety.

### TECHNICAL FIELD

This application is related to a folded absorbent article having a flexure-resistance of less than 450.0 grams-force, as well as a corresponding method for manufacturing such a folded absorbent article.

### BACKGROUND

In the field of absorbent articles, in particular open diapers and open baby diapers, there is a demand for providing a package of stacked absorbent articles that has a relatively symmetrical and stable outer shape for enabling improved handling and storing of the package and for providing an aesthetically attractive package.

Moreover, there is also a demand for providing a package of stacked absorbent articles in which a stack quality of the individual absorbent articles within the package is high, such that individual absorbent articles that are taken from the package has an appealingly appearance and are free from unintended folds that may have detrimental effect on absorption performance and leakage security.

Despite the activities in the field, there is still a demand for providing an improved package of stacked absorbent articles, and improved absorbent articles for enabling a better package, in terms of symmetrical, stable and aesthetically attractive packages, stack quality and quality of individual absorbent articles that are taken from the package.

There is continuous development towards thinner and more pliable absorbent articles because these characteristics generally results in improved wearing comfort, user fitting and discretion. However, thin and pliable absorbent articles are generally more problematic in terms of stacking and packaging of the stacked absorbent articles. Relatively thin and pliable absorbent articles have less internal structural rigidity and are therefore frequently unintentionally slightly misplaced or folded during the stacking and packaging process.

For example, one type of stacking and packaging process for absorbent articles involves placing a set of finished and bi-folded absorbent articles next to one another in individual compartments of a packaging tool, wherein the individual compartments are designed and oriented such that the set of absorbent articles resembles a stack. When each compartment is filled with an individual absorbent article, the packaging tool compresses the stack in the direction of the stack and pushes the stack of absorbent articles into a plastic bag that is subsequently closed to form a finished package.

However, the process step of inserting the stack of thin and pliable absorbent articles into the bag is problematic due to the relatively thin and pliable absorbent articles. In particular, the non-folded edge of a bi-folded absorbent article generally causes packaging problems due to the low internal stability. Bi-folded absorbent articles are absorbent articles having each of a first and second transversely opposed side edges folded onto a topsheet surface of the absorbent article, and subsequently being folded along a central fold line that extends perpendicular to the length direction of the flattened absorbent article, such that a topsheet of a rear portion after folding faced the topsheet of the front portion of the absorbent article. The non-folded edge of a bi-folded absorbent article is particularly structurally instable due to the lack of a folded edge at the non-folded end and due to the lack of a core at the front and rear waist edge, which form the non-folded edge of a bi-folded absorbent.

As a result, some absorbent articles may be slightly displaced with respect to the other absorbent articles in the stack because the thin and pliable absorbent articles tend to become deformed, folded, wrinkled, etc. instead of being properly pushed into the package during the step of pushing the stack of absorbent articles into a plastic bag.

This uncontrolled behavior of the individual absorbent articles during packaging process may result in reduced stack quality in terms of poor alignment of individual absorbent articles in the stack, less appealingly appearance due to unintended wrinkles or folds, increased interference with adjacent absorbent articles in the stack during removal of the absorbent article from the stack, detrimental effect of absorption capacity due to unintended folds of the absorbent core.

Moreover, stacking of thin and pliable absorbent articles tend to render the stack less stable, such that the overall shape of the finished and packaged package may deviate from a symmetrical shape. For example, the non-folded edge of a bi-folded thin and pliable absorbent article is particularly structurally instable and may result in a curved stacking axis due to the smaller thickness of the absorbent articles in the non-folded edge compared with the thickness of the folded edge.

The degree of pliability of an absorbent article can for example be assessed in terms of a flexure-resistance of an absorbent article. The flexure-resistance of an absorbent article corresponds approximately to the bending stiffness of the absorbent article, and thus gives a relatively reliable indication of the degree of flexibility of the absorbent article flexible and well it conforms to the various shapes of the body of the users when worn.

Thus, it would be desirable to provide an improved folded absorbent article having a relatively low flexure-resistance that enables improved packaging of a stack of folded absorbent articles into a consumer package, as well as an improved consumer package comprising a stack of folded absorbent articles with relative low flexure-resistance, in terms of symmetrical, stable and aesthetically attractive packages, stack quality and quality of individual absorbent articles that are taken from the package.

### SUMMARY

In order to achieve these technical objectives, this application provides, in several embodiments, a folded absorbent article, a package comprising such a folded absorbent article and a method for manufacturing such a folded absorbent article.

According to a first embodiment, there is provided a folded absorbent article comprising an absorbent core sandwiched between a liquid-permeable topsheet and a liquid-impermeable backsheet. The absorbent article comprises, in an unfolded and planar state, a longitudinal center line, a transverse center line, a longitudinal length extending from a front waist edge to a back waist edge, and a transverse length extending from a first side edge to a second side edge. The absorbent article further comprises a front portion, a back portion and a crotch portion, and fastening tabs located on each transverse side of the back portion for being releasably fastened to the front portion when the absorbent article is in a fastened position. The absorbent article has a flexure-resistance of less than 450.0 grams-force, specifically less than 300.0 grams-force, and more specifically less than about 230.0 grams-force and still more specifically less than 190.0 grams-force. Each transverse side of the absorbent article is folded along respective longitudinal fold line, such that each of first and second transversely opposed side edges are folded onto a topsheet surface of the absorbent article thereby forming a firstly folded absorbent article. The firstly folded absorbent article is folded along a first transverse fold line located in the region of the transverse center line thereby forming a secondly folded absorbent article. Finally, the secondly folded absorbent article is folded along a second transverse fold line located approximately in the middle between the transverse center line and a waist edge thereby forming a thirdly folded absorbent article.

According to a second embodiment, there is provided a method for manufacturing a folded absorbent article, the absorbent article comprising, in an unfolded and planar state, a longitudinal center line, a transverse center line, a longitudinal length extending from a front waist edge to a back waist edge, and a transverse length extending from a first side edge to a second side edge, said absorbent article further comprising a front portion, a back portion and a crotch portion, and fastening tabs located on each transverse side of the back portion for being releasably fastened to the front portion when the absorbent article is in a fastened position, the method comprising: forming an absorbent article having an absorbent core sandwiched between a liquid-permeable topsheet and a liquid-impermeable backsheet and a flexure-resistance of less than 450.0 grams-force, specifically less than 300.0 grams-force, and more specifically less than about 230.0 grams-force and still more specifically less than 190.0 grams-force; folding each transverse side of the absorbent article along respective longitudinal fold line, such that each of first and second transversely opposed side edges are folded onto a topsheet surface of the absorbent article thereby forming a firstly folded absorbent article; folding the firstly folded absorbent article along a first transverse fold line located in the region of the transverse center line thereby forming a secondly folded absorbent article; and folding the secondly folded absorbent article along a second transverse fold line located approximately in the middle between the transverse center line and a waist edge thereby forming a thirdly folded absorbent article.

One solution to the above-mentioned packaging problems is thus to apply quattro folding of the absorbent article because thereby the internal structural rigidity of the finished and folded absorbent article is significantly increased. In particular, the previous weak non-folded edge of a bi-folded absorbent article is substantially strengthened in terms of structural rigidity when applying quattro folding technique.

The term “quattro folding” herein refers to folding of an absorbent article such that a central core of the absorbent article has been folded twice along transverse folds lines, i.e. such that the absorbent article has a total of four overlapping main panels of the absorbent article. An unfolded article has a single main panel.

When each of first and second transversely opposed side edges have been folded onto a topsheet surface of the absorbent article to form the firstly folded absorbent article, and when the firstly folded absorbent article has been folded along the first transverse fold line located in the region of the transverse center line to form the secondly folded absorbent article, the secondly folded absorbent article may be deemed to have two overlapping sections of the absorbent article, i.e. defining a bi-folded absorbent article.

Furthermore, when the secondly folded absorbent article has been folded along the second transverse fold line located approximately in the middle between the transverse center line and the waist edge to form the thirdly folded absorbent article, the thirdly folded absorbent article may be deemed to have four overlapping sections of the absorbent article, i.e. defining a quattro folded absorbent article.

The quattro folded absorbent article forms a compact unit with high structural rigidity in all directions, since all edges of the quattro folded article in fact includes a folded edge. Both the unfolded front and back waist edge are located together with the folded edge of the first transverse fold line, the folded edge of the second transverse fold line is located at the oppositely located edge of the quattro folded article, and each of the side edges quattro folded article include a longitudinal fold line.

Moreover, the four overlapping sections of the absorbent article of approximately equal size efficiently stabilizes weak areas within the absorbent article, such as areas having particularly low flexure-resistance.

As a result of the increased structural rigidity of the quattro folded article the handling and position control of the folded absorbent article during the packaging process can be improved, and the risk for unsatisfactory alignment of individual absorbent articles in the stack, less appealingly appearance, interference with adjacent absorbent articles in the stack during removal, detrimental effect of absorption capacity, and unsymmetrical shape of the overall shape of the finished and packaged package, is reduced.

Moreover, the structure rigidity of the folded absorbent article is increased because the core is then present at all parts of the folded article to a larger degree, thereby also providing a thicker folded absorbent article.

In addition, quattro folding results in smaller and thicker folded absorbent articles, compared with bi-folded absorbent articles or non-folded absorbent articles. As a result, there is increased flexibility in terms of arranging the folded absorbent articles within the consumer package. This flexibility may for example be exploited for reducing the amount of package material required for the package with maintained number of folded absorbent articles within the package, or for providing a package with a larger front face surface area for increased brand exposure in the shelf of a warehouse. In addition, this flexibility may further be exploited for providing increased self-standing stability, or for increasing the packaging utilization on a standard size transportation pallet.

Moreover, in case of single-wrap of each individual folded absorbent article, i.e. where each folded absorbent article is wrapped in an individual wrapping, the amount of material required for each individual wrapping is reduced due to the shape of a quattro folded absorbent article is closer to the optimal form of a sphere compared with a bi-folded absorbent article that typically has a more flat, spread out and sheet-like outer geometry.

The smaller and thicker quattro folded absorbent articles also simplifies bring along of an individual absorbent article because it also fits into a relatively small purse or bag.

A thickness of the absorbent article in an unfolded state and in a region of the absorbent core may be less than 7 mm, specifically less than 6 mm, and more specifically less than 5 mm. A thinner absorbent product is typically more pliant than a thick product. Hence, the quattro folding is particularly advantageous for absorbent articles having a thickness in the area of the core of less than 7 mm, 6 mm or 5 mm. A method for measuring the thickness of the absorbent article is described in detail below.

A density of the absorbent article in the region of the absorbent core is higher than 0.16 g/cm³, specifically higher than 0.18 g/cm³, and more specifically higher than 0.20 g/cm³. A high density of the absorbent article in the region of the absorbent core is an indicator of relatively high SAP/pulp ratio and relatively low level of a voluminous fluff pulp in the core, because the SAP grains alone generally cannot create an integrated, stiff structure. In other words, a high density in the region of the absorbent core, at least for absorbent core having a relatively high SAP/pulp ratio, is an indicator of a relatively pliant absorbent article, which thus benefits from quattro folding. A method for measuring the density of the absorbent article is described in detail below.

An inner surface of the back portion may face an inner surface of the front portion of the secondly folded absorbent article, and an outer surface of the back portion may face an outer surface of the crotch portion of the thirdly folded absorbent article. This folding structure of an initially substantially planar absorbent article represents one variant out of a total of four available variants, each representing a unique final folding structure, of a quattro folded absorbent article that is first folded along a first central transverse fold line and subsequently folded along a second transverse fold line located approximately in the middle between the transverse center line and a waist edge, ignoring the longitudinal fold line along each transverse side of the absorbent article.

This particular variant, which has an inner surface of the back portion facing an inner surface of the front portion in the secondly folded absorbent article, ensures that the backsheet faces outwards and the topsheet, which will face the skin of the user, is better protected from dirt etc., thereby providing a more hygienic absorbent article.

Moreover, by subsequently selecting to have the outer surface of the back portion facing the outer surface of the crotch portion of the thirdly folded absorbent article, it is ensured that an outer front region of absorbent article remains visible on the quattro folded absorbent article. This is beneficial because the outer front region is typically used for informative printing, such as for example size of the absorbent article, and brand of the producer of the absorbent article. Hence, a consumer may still in a folded state of the thirdly folded absorbent article, i.e. quattro folded state, acquire relevant information about for example size, type and/or brand of the absorbent article, thereby reducing the need for unfolding the quattro folded article for acquiring this information.

A further advantage of having the inner surface of the back portion facing the inner surface of the front portion of the secondly folded absorbent article, and having the outer surface of the back portion facing the outer surface of the crotch portion of the thirdly folded absorbent article, is improved pre-forming of the absorbent article for better fit on the body of the user. A pre-formed absorbent article for better fit on the body of the user has typically slightly curved natural shape along the longitudinal center line with the topsheet facing towards a radially inner side of the curved shape. Such a curved natural shape is partly provided by folding the absorbent article along the first and second transverse fold lines as defined above.

Folding along the first transverse fold line to have the inner surface of the back portion facing the inner surface of the front portion of the secondly folded absorbent article creates a clear and desirable pre-forming of the article in crotch portion towards a having a curved shape with the topsheet located on the radially inner side.

Moreover, folding along the second transverse fold line to have the outer surface of the back portion facing the outer surface of the crotch portion of the thirdly folded absorbent article creates a pre-forming of the article towards a having a curved shape with the topsheet located on the radially inner side in the front portion of the article but the topsheet located on the radially outer side in the back portion of the article. However, since the absorbent core of the absorbent article typically extends closer to the front waist edge than the rear waist edge, and since the absorbent core may have a reduced thickness in back portion, desired pre-forming effect, i.e. having the topsheet located on the radially inner side, caused by folding along the second transverse fold line is relatively large in the front portion of the article, and the undesirable pre-forming effect, i.e. having the topsheet located on the radially outer side, at the second transverse fold line is relatively small in the back portion of the article. To conclude, the specific quattro folding results in a desirable pre-forming effect on the article in the front and crotch portion, while the undesirable pre-forming effect in the back portion generally is relatively weak due to reduced thickness or lack of absorbent core in the back portion.

Still a further advantage of the above-defined specific quattro folding is that folding of the article along second transverse fold line results in a smaller radius fold at the back portion and a larger radius fold at the front portion since the front portion of the article surrounds the back portion, as seen from a lateral side of article. This is advantageous because the back portion of the article typically has a smaller thickness than the front portion, partly due to the aforementioned forwards located core and reduced thickness of the absorbent core in the back portion, thereby enabling the back portion to more easily and with less risk for damages to the absorbent core have a smaller radius fold than the front portion, and thus providing an overall thinner folded absorbent article.

The absorbent core may be constituted of one single core layer. This enables simplified manufacturing of the absorbent article.

The absorbent core may have a generally rectangular shaped, as seen from a top of the absorbent article in a flat state. This enables less scrap material when cutting the absorbent core from a continuous strip of absorbent material.

The absorbent article may be an open baby diaper absorbent article. These type of articles are generally relatively small and thereby particularly difficult to pack high stack quality and without unintended folds and wrinkles.

The absorbent core may comprise about 60-100 wt % SAP. If the absorbent core comprises less than 100 wt % SAP the remaining material may for example be primarily pulp material, or the remaining material may for example be only pulp material. A relatively high SAP/pulp ratio indicates a thin absorbent article and thus a relatively pliant absorbent article. Hence, quattro folding is particularly advantageous when the absorbent core comprises about 60-100 wt % SAP.

The absorbent core may in some example embodiments comprise one, two or more primarily longitudinally extending channels located at least in the crotch portion of the absorbent core. One or more longitudinally extending channels may in certain designs improve insulting fluid distribution along the length of the channel, such that a wider area of the core may be used for fluid absorption and a better utilization of the absorbent capacity of the core may be accomplished. The channels may also provide increased fluid acquisition speed. However, one or more primarily longitudinally extending channels located at least in the crotch portion of the absorbent core generally results in further reduced structural rigidity and increased pliability of the absorbent article, thereby further emphasising the advantage of applying quattro folding.

In another embodiment, a consumer package is provided comprising a stack of folded absorbent article as described above. Packaging of particularly plaint and low flexure-resistant absorbent articles is simplified after quattro folding of the absorbent articles.

The package may for example comprise at least two parallel stacks, specifically at least three parallel stacks, and more specifically at least four parallel stacks of folded absorbent articles located side-by-side. As described above, increased number of parallel stacks enables outer package dimensions that are less narrow and more resembling a cubical shape, thereby enabling increased front face surface for displaying size and brand, as well as providing a more self-stable article, especially with having relatively low number of articles in each package.

Further features of, and advantages with, the present disclosure will become apparent when studying the following description. The skilled person realize that different features of the present disclosure may be combined to create embodiments other than those described in the following, without departing from the scope of the present disclosure.

### BRIEF DESCRIPTION OF THE DRAWINGS

The features and advantages of the invention will be appreciated upon reference to the following drawings. The accompanying drawings, which are incorporated in and constitute a part of this specification, illustrate one or more embodiments of the invention and, together with the general description given above and the detailed description given below, explain the one or more embodiments of the invention.

FIG. 1 is a top perspective view of a bi-folded absorbent article.

FIG. 2 is a top perspective view of the absorbent article of FIG. 1 when axial compression forces are exerted in the folded absorbent article.

FIG. 3 is a top view of an absorbent article in a flat state.

FIG. 4 is a top view of the absorbent article of FIG. 3 after folding into a firstly folded absorbent article according to one embodiment.

FIG. 5 is a top view of the absorbent article of FIG. 3 after folding into a secondly folded absorbent article.

FIG. 6 is a top view of the absorbent article of FIG. 3 after folding into a thirdly folded absorbent article.

FIG. 7 is a top perspective view of an absorbent article in a flat state corresponding to FIG. 3.

FIG. 8 is atop perspective view of the absorbent article of FIG. 7 after folding into a firstly folded absorbent article.

FIG. 9 is a top perspective view of the absorbent article of FIG. 7 after folding into a secondly folded absorbent article.

FIG. 10 is a top perspective view of the absorbent article of FIG. 7 after folding into a thirdly folded absorbent article.

FIG. 11 is a side view of an absorbent article in quattro folded state.

FIG. 12 is a perspective view of an example of a pre-formed natural state of the article of FIG. 11 after unfolding.

FIG. 13 is a perspective view of an example of open diaper in an open state that may be folded according to one embodiment.

FIG. 14 is a top perspective view of the diaper of FIG. 13 in a closed state.

FIG. 15 is a top view of an example embodiment of a package of folded absorbent articles according to another embodiment.

FIG. 16 is a top view of a further example embodiment of a package of folded absorbent articles according to yet another embodiment.

FIG. 17 is a side cross-section of the package of FIG. 16, taken along line A-A in that FIG. 16.

FIG. 18 is a side cross-sectional view of the package of FIG. 17 in an open state.

FIG. 19 is a top view of the absorbent article in a flat state and with a plurality of samples illustrated.

### DETAILED DESCRIPTION

The present folded absorbent article and associated package will now be described more fully hereinafter with reference to the accompanying drawings, in which exemplary embodiments of the disclosure are shown. The folded absorbent article and associated package may, however, be embodied in many different forms and should not be construed as limited to the embodiments set forth herein; rather, these embodiments are provided for thoroughness and completeness. Like reference characters refer to like elements throughout the description. The drawings are not necessarily to scale and certain features may be exaggerated in order to better illustrate and explain the exemplary embodiments of the present disclosure.

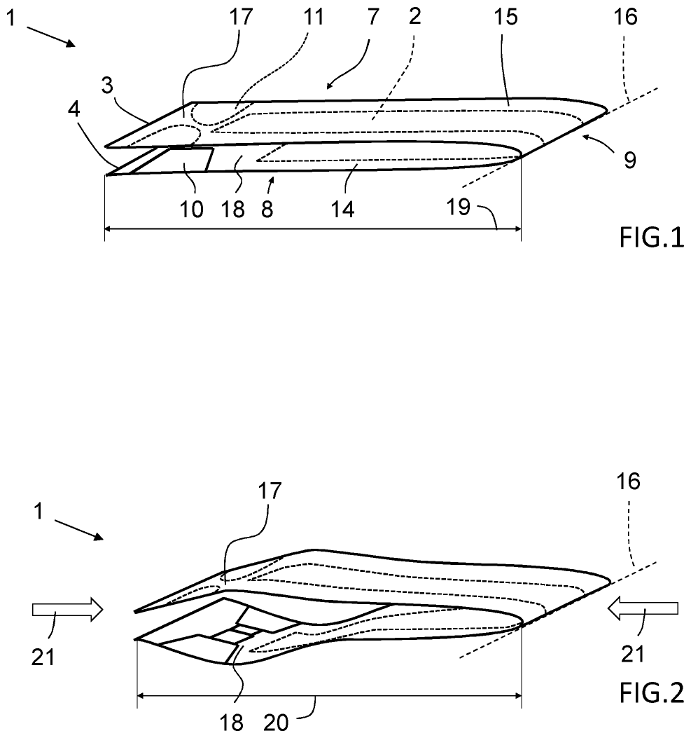

With initial reference to FIG. 1 there is shown a schematic 3D-view of an example embodiment of a bi-folded open absorbent article 1 comprising an absorbent core 2, a liquid-permeable topsheet 14 and a liquid-impermeable backsheet 15, a front waist edge 3 and a back waist edge 4. The absorbent article 1 further comprising a front portion 7, a back portion 8, a crotch portion 9, and fastening tabs 10 located on the back portion 8 and side panels 11 on the front portion 7.

The topsheet 14 is arranged at the surface of the article, i.e. at the side facing the wearer, whereas the backsheet 15 is arranged at the underside of the article 1. Furthermore, both the topsheet 14 and the backsheet 15 may extend laterally outside of the absorbent core 2 along the entire perimeter of the article 1.

The absorbent article has been folded along a centrally located first transverse fold line 16 to have two overlapping sections 17, 18 of the absorbent article 1 of approximately equal size, thereby forming the bi-folded absorbent article 1 having a first longitudinal length 19.

The absorbent article is of a type that has a relatively low flexure-resistance. This may be particularly problematic during manufacturing and packaging of individual absorbent articles 1 in stacks enclosed by a bag.

For example, upon placing a set of individual articles 1 in stack and subsequently compressing the stack and pushing the stack into an empty bag the risk for damages to the absorbent articles 1 in terms of unintended folds and wrinkles to the absorbent articles is increased when the absorbent articles have relatively low flexure-resistance and thus relatively low internal structural strength.

Moreover, the manufacturing step of placing a set of individual articles 1 in stack in an aligned manner to provide a high stack quality may also be problematic because the relatively pliable absorbent articles 1 may more easily deform during handing by the mechanical manufacturing equipment that packages stacks of individual absorbent articles in bags.

For example, one type of stacking and packaging process for absorbent articles involves placing a set of finished and bi-folded absorbent articles next to one another in individual compartments of a packaging tool, wherein the individual compartments are designed and oriented such that the set of absorbent articles resembles a stack. When each compartment is filled with an individual absorbent article the packaging tool compresses the stack in the direction of the stack and pushes the stack of absorbent articles into a plastic bag that is subsequently closed to form a finished package.

However, some individual absorbent articles of the stack may be slightly deformed upon being placed in the individual compartments, such as for example schematically illustrated in FIG. 2, where the bi-folded absorbent article 1 has become slightly compressed in a longitudinal direction to have a second longitudinal length 20 that is smaller than the first longitudinal length 19. Such deformation may for example be caused by an axial force 21 that is exerted on the bi-folded absorbent article 1 in the longitudinal direction. This has a detrimental effect on stack quality because the alignment of individual absorbent articles 1 within the stack will then be degraded.

Consequently, some absorbent articles may be positioned slightly offset with respect to the other absorbent articles in the stack and this typically causes some absorbent articles to be unintentionally folded at an edge of the article. The uncontrolled behavior of the individual absorbent articles during packaging process caused by the relatively low flexure-resistance thus potentially results in reduced stack quality, less appealingly appearance due to unintended wrinkles or folds, and possibly even detrimental effect of absorption capacity due to unintended folds of the absorbent core.

The solution to this problem is to apply so-called quattro folding on the absorbent article for improving the structural rigidity of the folded absorbent article and thereby improving handling and packaging quality of the absorbent article. The new folding according to the disclosure is particularly suitable for solving the problem of handling of absorbent articles with a relatively low flexure-resistance because the additional folding has a strengthening effect on the folded absorbent article 1.

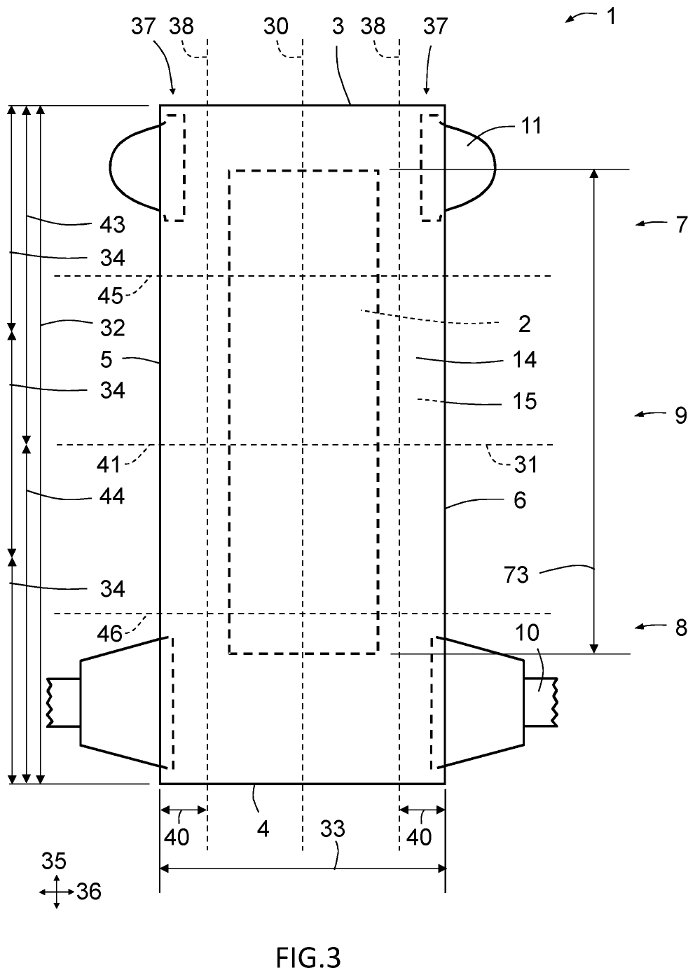

An example embodiment of the absorbent article 1 will now be described in detail with reference to FIG. 3. The absorbent article 1 comprises an absorbent core 2 sandwiched between a liquid-permeable topsheet 14 and a liquid-impermeable backsheet 15. The absorbent article comprises, in the illustrated unfolded and planar state, a longitudinal center line 30, a transverse center line 31, a longitudinal length 32 extending from a front waist edge 3 to a back waist edge 4, and a transverse length 33 extending from a first side edge 5 to a second side edge 6 in a transverse direction 36.

The absorbent article further comprises a front portion 7, a back portion 8 and a crotch portion 9, each having the same length 34 in the longitudinal direction 35. The absorbent article 1 further comprises fastening tabs 10 located on each transverse side edge 5, 6 of the back portion 8 for being releasably fastened to the front portion 7 when the absorbent article is in a fastened position on a user, and side panels 11 on the front portion 7.

The absorbent core 2 may further comprise one, two, or more primarily longitudinally extending channels (not showed) located at least in the crotch portion 9 of the absorbent article 1 for improved fluid acquisition and distribution in the absorbent core 2.

The absorbent core shown in FIG. 1 has a substantially rectangular design. However, the disclosure is not limited to this design but may be formed in generally any geometric form within the scope of the disclosure. Moreover, the absorbent core may be constituted of one single core layer, or two or more stacked core layers.

The absorbent article 1 has a flexure-resistance of less than 450.0 grams-force, specifically less than 300.0 grams-force, and more specifically less than about 230.0 grams-force and still more specifically less than 190.0 grams-force. In other words, the absorbent article according to the present disclosure is highly flexible, bendable and pliant, making it very comfortable for the user to wear, as well as providing high leakage security due to its high capacity to adapt the specific form of each unique user.

The flexure-resistance of the absorbent article is measured by peak bending stiffness. Peak bending stiffness is determined by a test, which is modelled after the ASTM D 4032-82 CIRCULAR BEND PROCEDURE, the procedure being modified and performed as follows.

The CIRCULAR BEND PROCEDURE is a simultaneous multi-directional deformation of a material in which one face of a sample becomes concave and the other face becomes convex. The CIRCULAR BEND PROCEDURE gives a force value related to flexure-resistance, simultaneously averaging stiffness in all directions.

The apparatus necessary for performing the CIRCULAR BEND PROCEDURE according to the disclosure is a modified Circular Bend Stiffness Tester, having the following parts:

A smooth-polished steel plate platform, which is 102.0×102.0×6.35 mm having an 18.75 mm diameter orifice. The lap edge of the orifice should be at a 45-degree angle to a depth of 4.75 mm.

A plunger having an overall length of 72.2 mm, a diameter of 6.25 mm, a ball nose having a radius of 2.97 mm and a needle-point extending 0.88 mm therefrom having a 0.33 mm base diameter and a point having a radius of less than 0.5 mm, the plunger being mounted concentric with the orifice and having equal clearance on all sides. Note that the needle-point is merely to prevent lateral movement of the test sample during testing. Therefore, if the needle-point significantly adversely affects the test sample (for example, punctures an inflatable structure), than the needle-point should not be used. The bottom of the plunger should be set well above the top of the orifice plate. From this position, the downward stroke of the ball nose is to the exact bottom of the plate orifice.

A force-measurement gauge and more specifically an inverted compression load cell. The load cell may have a load range of from 0.0 to 2000.0 grams-force.

An actuator, such as a conventional tensile tester that can be adapted for compression, having the inverted compression load cell. Suitable conventional tensile testers are available for example from Instron or Lloyd corporations.

In order to perform the procedure for this test, as explained below, five representative absorbent articles are used. The five absorbent articles may be selected randomly from a consumer package. Alternatively, five absorbent articles are selected randomly from a line of 40 adjacent absorbent articles on a factory production line.

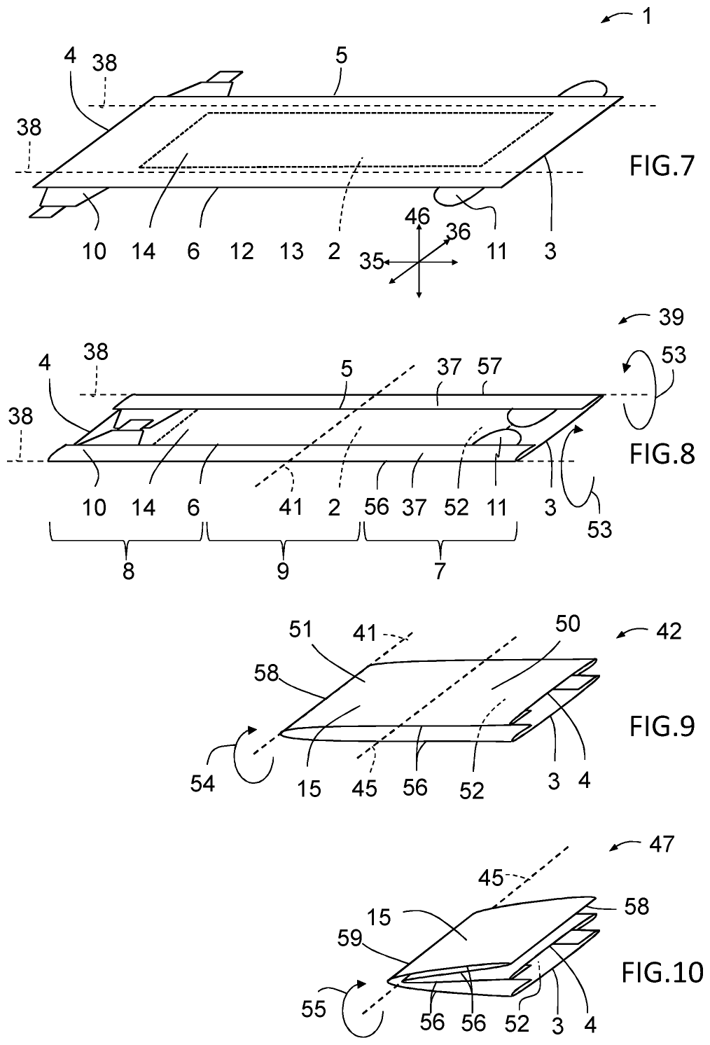

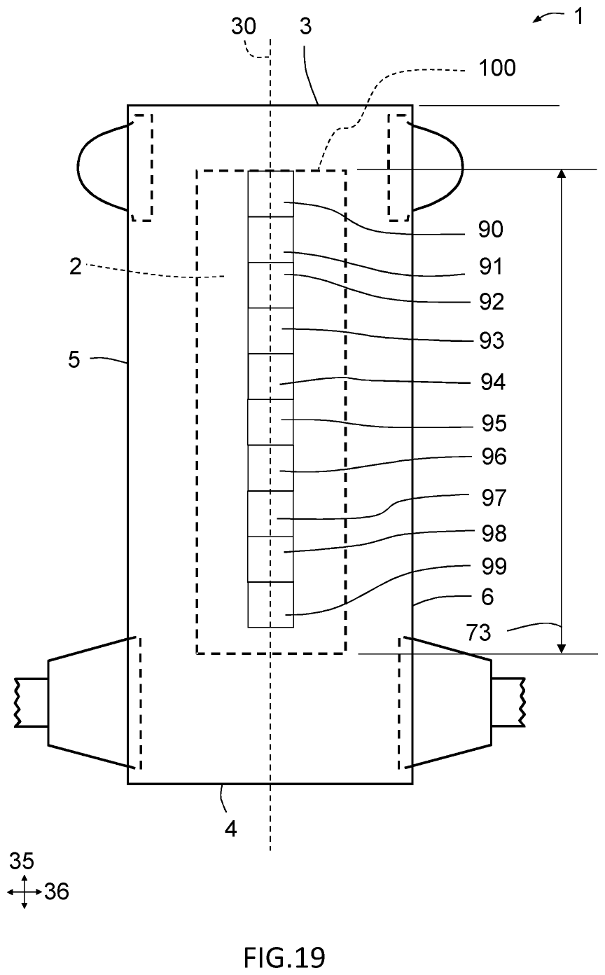

The flexure-resistance of the absorbent article 1 is measured in an area of the absorbent core 2 of the absorbent article 1, i.e. an area of the absorbent article 1 overlapping the absorbent core 2 in a thickness direction 46 of the absorbent article 1 in an unfolded and planar state as showed in FIG. 3 and FIG. 7. The method for measuring the flexure-resistance of the absorbent article is herein described with reference to FIG. 19, which shows the absorbent article 1 in a flat state and with a row adjacent individual test samples 90-98 of the absorbent article 1. According to this example embodiment, the number of samples, hereinafter denoted “Y”, is 10 samples.

The method for measuring the flexure-resistance of the absorbent article comprises:

cutting off or neutralizing all elastic elements in the absorbent article 1,

placing the absorbent article 1 in a flat and smooth state,

punching out adjacent samples 90-99 of the absorbent article in the area of the absorbent core 2 starting from the front end 100 of the area of the absorbent core 2, which samples measure 3.75×3.75 cm and are located centered along the longitudinal center line 30.

Consequently, as an example, an article with a 40 cm long area of the absorbent core would thus yield 10 individual samples and with an extra 2.5 cm piece in the back that is discarded. A short absorbent core in the longitudinal direction 35 would of course result in a smaller number of individual samples, and oppositely.

From each of the five absorbent articles of the same type to be tested, some number “Y” of 37.5×37.5 mm test samples are thus cut. Hence, the test person should have five sets of test samples, each set including “Y” number of samples.

The flexure-resistance is determined after 24 hours conditioning by placing the punched samples free and bare in a laboratory environment set to 23° C. and 50% relative humidity. The remaining part of the test method is performed in the same environment.

The test samples should not be excessively folded or bent by the test person, and the handling of samples should be kept to a minimum and to the edges to avoid affecting flexural-resistance properties.

The procedure for the CIRCULAR BEND PROCEDURE according to the disclosure is as follows. The test plate is levelled. The plunger speed is set at 50.0 cm per minute per full stroke length. A sample is centered on the orifice platform below the plunger such that the body surface of the sample is facing the plunger and the garment surface of the sample is facing the platform. Body surface herein means the surface of the sample that is intended to face the body of a user, and garment surface herein means the surface of the sample that is intended to face the garment of a user.

The force-measurement gauge is checked and adjusted to zero, if necessary. The plunger is actuated. Touching the sample during the testing should be avoided. The maximum force reading to the nearest grams-force is recorded. The above steps are repeated until all Y number of samples of each of the five identical type absorbent articles have been tested.

The peak bending stiffness, i.e. flexure-resistance, for each sample is the maximum force reading for that sample. The measured flexure-resistance of each set of Y number of tested samples is thereafter averaged to receive an average flexure-resistance value for each of the five absorbent articles. Thereafter, the five averaged flexure-resistance values are averaged to receive a final flexure-resistance value representing the specific type of the absorbent article.

A test for determining various relevant parameters of five different absorbent articles have been performed and the test result in presented in Table 1 below. Products A-D are open baby diapers and product E is an adult open incontinent diaper.

The flexure-resistance of the products A-E has been determined using the test method described above and is provided in the column “Parameter 5” (P5). Both product A and B clearly has a very low flexure resistance and would thus particularly benefit from Quattro folding before packaging in a consumer package for increasing the quality of the package and the absorbent products therein, as well as simplifying the packaging process.

TABLE 1
part 1

| P1 | P2 | P3 | P4 | P5 | P6 |
|---|---|---|---|---|---|
| A | 3.7 | 0.2 | 720 | 182 | Single core |
| B | 3.5 | 0.21 | 749 | 185 | Single core |
| C | 4.9 | 0.15 | 723 | 494 | SAP laminate (SAP |
| | | | | | between two layers |
| | | | | | of SMS) + Curly |
| | | | | | fibre core on top |
| D | 5 | 0.18 | 895 | 529 | Stacked dual core |
| E | 5.5 | 0.14 | 768 | 874 | Stacked dual core |

TABLE 1
part 2

| P1 | P7 | P8 | P9 | P10 | P11 |
|---|---|---|---|---|---|
| A | 2 × 12 gsm | 20 gsm SB | 50 gsm | 78 wt % SAP | 2 × 12 gsm |
| | SMS | Nonwoven | Thermobonded | | SMS |
| B | 2 × 12 gsm | 20 gsm SB | 50 gsm | 78 wt % SAP | Film/NW laminate |
| | SMS | Nonwoven | Thermobonded | | 15/14 gsm |
| C | 2 × 11 gsm | 15 gsm SB | 45-50 gsm binder | 100 wt % SAP + | Film/NW laminate |
| | SMS | Nonwoven | bonded loft | glue | 15/14 gsm |
| D | 10 gsm + 8 | 20 gsm SB | 50 gsm | 63 wt % SAP | Film/NW laminate |
| | gsm SMS | Nonwoven | Thermobonded | | 15/14 gsm |
| E | No core | 15 gsm SB | 29 gsm aperture | 22 wt % SAP | Film/NW laminate |
| | cover | Nonwoven | film | | 15/14 gsm |

### Parameters of Table 1:

P1: Product type
P2: Thickness [mm]
P3: Density [g/cm³]
P4: Basis weight [gsm]
P5: Flexure-resistance [gf]

### P6: Core

P7: Core cover

### P8: Topsheet

P9: Loft/acquisition layer
P10: SAP/Pulp ratio

### P11: Backsheet

### SMS=Spunbond/Meltblown/Spunbond

### SB=Spunbond Nonwoven

Further factors that generally are closely related and having an effect on the pliability and level of flexure-resistance of the absorbent article 1 is thickness and density of the absorbent article 1 in the area of the absorbent core 2, as well as SAP/Pulp ratio of the absorbent core.

A thinner absorbent article 1 typically results in reduced internal structural strength, thereby making the absorbent article 1 more floppy and pliable. Hence, a relative thin absorbent article 1 is an indication that the absorbent article 1 has a relatively low level of flexure resistance.

Similarly, a relatively thin absorbent article 1 typically has a relatively high SAP/Pulp ratio, i.e. having generally a larger content of SAP than Pulp, in order to maintain the absorption capacity of the relatively thin absorbent article. However, SAP typically has a higher density than fluff pulp. Consequently, there is a relationship linking high density with relatively thin absorbent articles having relatively low-level flexure-resistance. In other words, a higher density may generally be deemed functionally linked with reduced level of flexure-resistance, at least in combination with a relative thin absorbent article.

According to the disclosure, the thickness of the absorbent article 1 in a region of the absorbent core 2 may be less than 7 mm, specifically less than 6 mm, and more specifically less than 5 mm. Theses ranges defining an increasingly thinner absorbent article generally correspond to gradually reduced flexure-resistance of the absorbent article 1.

The thickness of the absorbent article 1 is measured in the area of the absorbent core 2 of the absorbent article 1. The method for measuring the thickness of the absorbent article is based on the same preparation of test samples collected from five absorbent articles as described above with reference to the test method for determining the flexure-resistance of the absorbent article 1.

Consequently, method for measuring the thickness of the absorbent article is described with reference to FIG. 19 and comprises:

cutting off or neutralizing all elastic elements in the absorbent article 1,

placing the absorbent article 1 in a flat and smooth state,

punching out adjacent samples 90-98 of the absorbent article in the area of the absorbent core 2 starting from the front end 99 of the area of the absorbent core 2, which samples measure 3.75×3.75 cm and are located centered along the longitudinal center line 30.

The thickness is determined after 24 hours conditioning by placing the punched samples free and bare in a laboratory environment set to 23° C. and 50% relative humidity. The remaining part of the test method is performed in the same environment.

The thickness of the individual samples is measured under a 2.5 kPa (kilopascal) pressure. The method for measuring the thickness includes: Lowering a circular foot with a 35 mm diameter slowly over a center point of the sample and determine the thickness when the foot has rested on the sample for three seconds. The thickness of the absorbent article is subsequently determined by averaging the determined thickness of each sample, i.e. by calculate the average thickness for the samples.

According to the disclosure, the density of the absorbent article 1 in the region of the absorbent core is higher than 0.16 g/cm³, specifically higher than 0.18 g/cm³, and more specifically higher than 0.20 g/cm³.

The density of the absorbent article 1 is measured in the area of the absorbent core 2 of the absorbent article 1. First, an average sample weight is determined. This is performed by weighing all the 3.75×3.75 cm samples from the absorbent article (all together) on a balance (accurate to 0.001 g), and divide by the number of samples.

Finally, the average density (g/cm³) is calculated by dividing the average sample weight (g) by the product of the sample area (14.06 cm²) multiplied with the average sample thickness in (cm), i.e. the average density=average sample weight/(14.06×average sample thickness).

The thickness and density measurements are performed on five absorbent articles selected randomly from a consumer package. Alternatively, five absorbent articles are selected randomly from a line of 40 adjacent absorbent articles on a factory production line. The overall average, i.e. the average of the averages from the five individual absorbent articles, represents the thickness and density of any specific absorbent article model in the context of this disclosure.

Considering that the method for acquiring the test samples for determining the flexure-resistance of the absorbent article is identical to the method for acquiring the test samples for determining thickness and density of the absorbent article, the same samples that are used for determining the flexure-resistance of the absorbent article may be used for determining thickness and density of the absorbent article. In such case thickness and/or density of the samples are preferably determined first and the flexure-resistance thereafter, because the flexure-resistance test may result in permanent damages to the test samples.

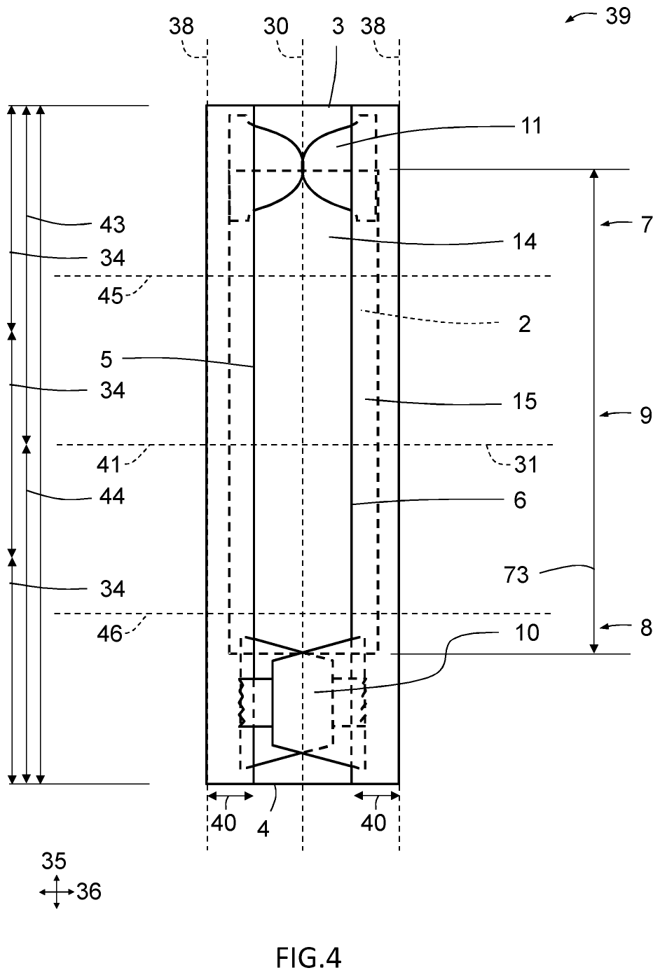

A folding of the absorbent article shown in FIG. 3 will now be described in detail with reference to FIGS. 4-6, which schematically shows different folding stages of the absorbent article 1.

In a first folding step each transverse side 37 of the absorbent article 1 is folded along respective longitudinal fold line 38, such that each of first and second transversely opposed side edges 5, 6 are folded onto a topsheet surface of the absorbent article 1 thereby forming a firstly folded absorbent article 39. The firstly folded absorbent article 39 is schematically illustrated in FIG. 4.

The longitudinal fold lines 38 may be located at the same distance from each transverse side edge 5, 6, respectively. A transverse length 40 between each longitudinal fold line 38 and the respective transverse side edge 5, 6 may be 5-30% of the transverse length 33 between the side edges 5, 6 of the unfolded absorbent article. Moreover, the longitudinal fold lines 38 are preferably located transversally outside of the absorbent core 2 for avoiding unnecessary folding of the absorbent core 2.

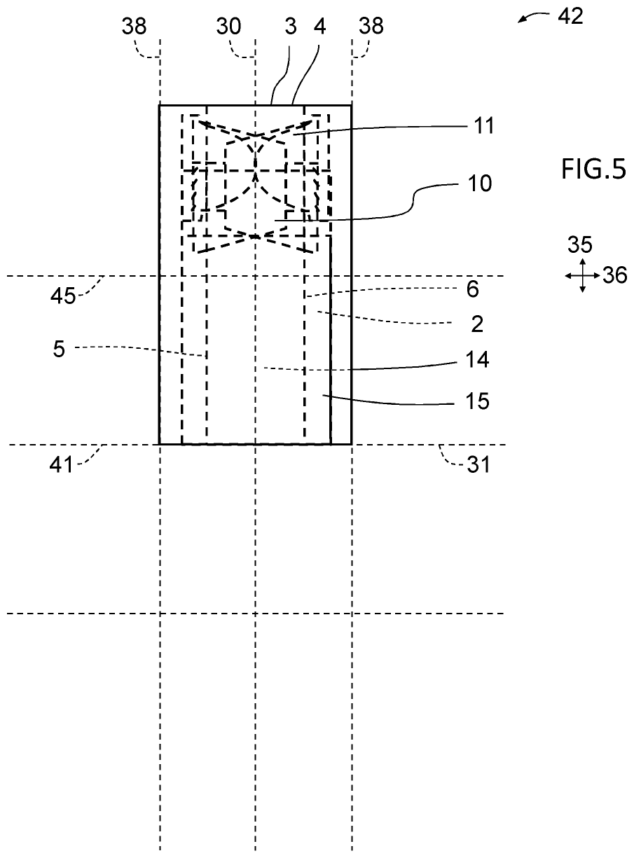

Thereafter, in a second folding step, the firstly folded absorbent article 39 is folded along a first transverse fold line 41 located in the region of the transverse center line 31 thereby forming a secondly folded absorbent article 42. The secondly folded absorbent article 42 is schematically illustrated in FIG. 5.

The secondly folded absorbent article 42 is considered to have two overlapping main sections of the absorbent article 1, namely a front section 43 and a back section 44. Having the first transverse fold line 41 located essentially aligned with the transverse center line 31 generally results in a minimal size of the secondly folded absorbent article 42 in the longitudinal direction. However, the first transverse fold line 41 may alternatively be located in the region of the transverse center line 31 that is slightly longitudinally offset from the transverse center line 31 for any reason. For example, the first transverse fold line 41 may displaced up to about 10% of the total length 32 of the absorbent article 1 from the transverse center line 31.

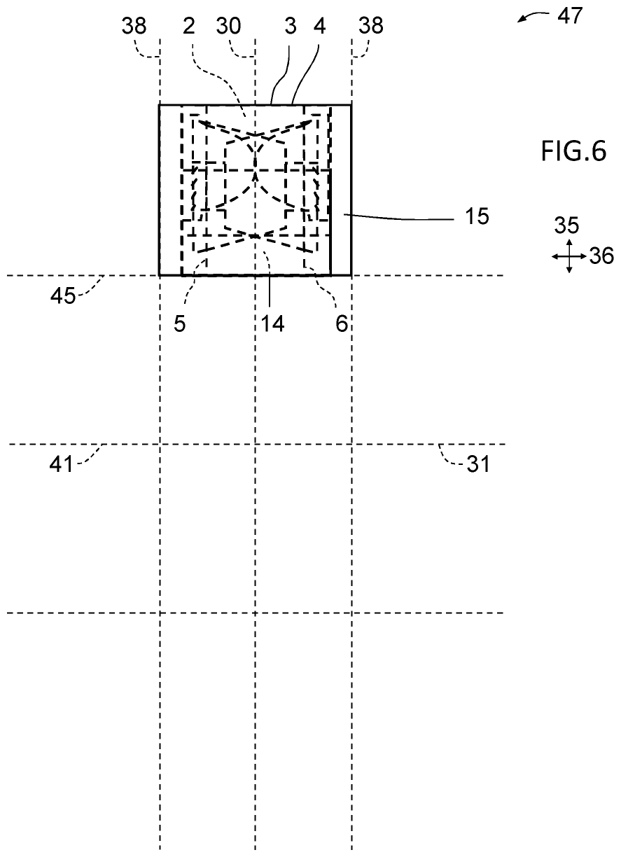

Thereafter, in a third folding step, the secondly folded absorbent article 42 is folded along a second transverse fold line 45 located approximately in the middle between the transverse center line 31 and a waist edge 3 thereby forming a thirdly folded absorbent article 47. The thirdly folded absorbent article 47 is schematically illustrated in FIG. 6.

The thirdly folded absorbent article 47 is considered to have four overlapping main sections of the absorbent article 1, namely twice the front section 43 and twice the back section 44. For this reason, the thirdly folded absorbent article 47 is considered to have a so-called quattro folding.

In certain example embodiments, the absorbent core comprises at least one channel (not showed) located at least in the crotch portion 9 of the absorbent article 1 for enabling improved fluid acquisition and fluid distribution in the absorbent core 2. The at least one channel may have a length which is between 5-50%, specifically 10-50%, and more specifically 28-38%, of the total length 32 of the absorbent article 1.

Moreover, that at least one channel may have a length which is between 10-60%, specifically between 20-60%, and more specifically between 30-50%, of a length 73 of the absorbent core 2.

In addition, a distance between the front waist edge 3 of the absorbent article 1 and a front edge of the at least one channel may be between 15-40%, and specifically between 22-25%, of a total length 32 of the absorbent article 1.

In certain example embodiments, the at least one channel may have a width of at least 3 mm, specifically at least 4 mm, and more specifically at least 5 mm, as seen in the transverse direction 36.

In certain example embodiments, the at least one channel may have are generally straight shape and oriented with a longitudinal axis generally parallel to the longitudinal center line 30 of the absorbent article 1.

If the absorbent core comprises two channels, the two channels may have the same length, width and form. Moreover, the two channels may be position in a central region of the absorbent article 1 and symmetrically on each side of the longitudinal center line 30.

The at least one channel may have various design and compositions. For example, the at least one channel may constitute sections of the absorbent core 2, which are generally free from absorbent material. This may for example be realized by cutting out at least one channel in a finished absorbent core. Alternative, the at least one channel may be obtained through manufacturing the absorbent core 2 involving a mat forming process during which absorbent material is omitted from the at least one area, which correspond to the at least one channel. In this manner, no absorbent material will be present in the at least one channel.

Alternatively, the at least one channel may constitute sections of the absorbent core 2, which have a thinner layer of absorbent material than adjacent areas of the absorbent core 2, as measured in the thickness direction 46.

The length and position of the at least one channel, and the position of the second transverse fold line 45 of the quattro folded absorbent article may be selected such that the second transverse fold line 45 of the quattro folded absorbent article does not intersect the at least one channel. Thereby, damages to the at least one channel caused by the folding along the second transverse fold line 45 is avoided

For better describing the folding sequence of FIG. 3-6 the substantially the same folding sequence is illustrated in a schematic 3D-view of the absorbent article in FIGS. 7-10. The absorbent article of FIGS. 3-6 has a similar structure, shape and composition as the absorbent article illustrated in FIGS. 7-10.

FIG. 7 shows the absorbent article 1 in an unfolded and planar state having the absorbent core 2 sandwiched between the topsheet 14 and backsheet 15, the front waist edge 3, back waist edge 4, and first and second side edges 5, 6.

The longitudinal direction 35, the transverse direction 36 and a thickness direction 46 of the absorbent article 1 are indicated in FIG. 7, wherein the thickness direction 46 extends perpendicular to the longitudinal and transverse direction 35, 36.

In a first folding step, the transverse sides 37 of the absorbent article 1 are folded along the longitudinal fold lines 38 to form the firstly folded absorbent article 39, as illustrated in FIG. 8. The folding operation is illustrated by first folding arrows 53, which show the folding of the transverse sides 37 along the longitudinal fold lines 38. The transverse side edges 56, 57 of the firstly folded absorbent article 39 are substantially coaxial with the longitudinal folds lines 38.

In the subsequent second folding step, the firstly folded absorbent article 39 is folded along the first transverse fold line 41 located in the region of the transverse center line to form the secondly folded absorbent article 42, which is illustrated in FIG. 9. The folding operation is illustrated by second folding arrow 54 that show the folding of the firstly folded absorbent article 39 along the first transverse fold line 41. The folded edge 58 in the crotch portion 9 is substantially coaxial with the first transverse fold line 41.

As clearly illustrated in FIG. 9, the topsheet 14 of the back portion 8 has been folded to face the topsheet 14 of the front portion 7 in the secondly folded absorbent article 42. Thereby, the backsheet 15 faces outwards of the secondly folded absorbent article 42 and the topsheet 14, which will face the skin of the user, is better protected from dirt etc., thereby providing a more hygienic absorbent article 1.

Moreover, this specific folding additionally provides a desirable pre-forming of the absorbent article 1, such that the absorbent article upon unpacking from the package has a curved natural shape with the topsheet facing a radially inner surface of such a curved natural shape.

Finally, in the third folding step, the secondly folded absorbent article 42 is folded along a second transverse fold line 45 located approximately in the middle between the transverse center line and the waist edge 3 thereby forming the thirdly folded absorbent article 47, which is illustrated in FIG. 10. The folding operation is illustrated by third folding arrow 55 that show the folding of the secondly folded absorbent article 42 along the second transverse fold line 45.

As discussed above, folding of the absorbent article 1 such that the inner surface of the back portion 8 faces the inner surface of the front portion 7 of the secondly folded absorbent article 42, and such that the outer surface 50 of the back portion 8 faces the outer surface 51 of the crotch portion 9 of the thirdly folded absorbent article 47, provides improved pre-forming of the absorbent article, wherein the pre-forming involves providing the unfolded absorbent article 1 with a step-wise curved shape for better fit on the curved natural shape of the body of the user between the stomach and back. The folds of the absorbent core 2 along both the first transverse fold line 41 and the forward of the second transverse fold line 45 results in a step-wise curved shape of the unfolded absorbent article with the topsheet 14 facing radially inwards in the curve, thereby providing improved fitting, comfort and leakage security of the article when carried by a user.

The circumferential edges of the quattro folded absorbent article of FIG. 10 are: on a first side the folded edge 59 located approximately in the middle between the transverse center line 31 and a waist edge 3 and substantially coaxial with the second transverse fold line 45, on a second side four layers of the transverse side edges 56 of the firstly folded absorbent article 39, on a third side the folded edge 58 in the crotch portion 9 that is located substantially overlapping with the front waist edge 3 and back waist edge 4, and on the fourth side the four layers of the transverse side edges 57 of the firstly folded absorbent article 39. Hence, all sides of the quattro folded absorbent article shown in FIG. 10 comprises folded portions of the absorbent article 1, thereby strengthening the absorbent article 1. The additional strengthening effect resulting from having four sections of the absorbent article in overlapping arrangement efficiently stabilizes areas of the absorbent article with low flexure-resistance and enables the folded absorbent article to be properly handled by the mechanical manufacturing and packaging devices with maintained proper position control of each individual absorbent article, such that high stacking quality is accomplished and packaging can be performed without having unacceptable amount of unintentional folds and wrinkles of the absorbent articles.

As clearly shown in FIGS. 9 and 10, folding of the secondly folded absorbent article 42 of FIG. 9 along the second transverse fold line 45 to form the thirdly folded absorbent article 47 involves folding the secondly folded absorbent article 42 such that an outer surface 50 of the back portion 8 comes to face an outer surface 51 of the crotch portion 9. This way of folding the secondly folded absorbent article 42 has several advantages. For example, the outer front region 52 of absorbent article 1 remains visible on the quattro folded absorbent article shown in FIG. 10. This is beneficial because the outer front region is typically used for informative printing, such as for example size of the absorbent article, and brand of the producer of the absorbent article.

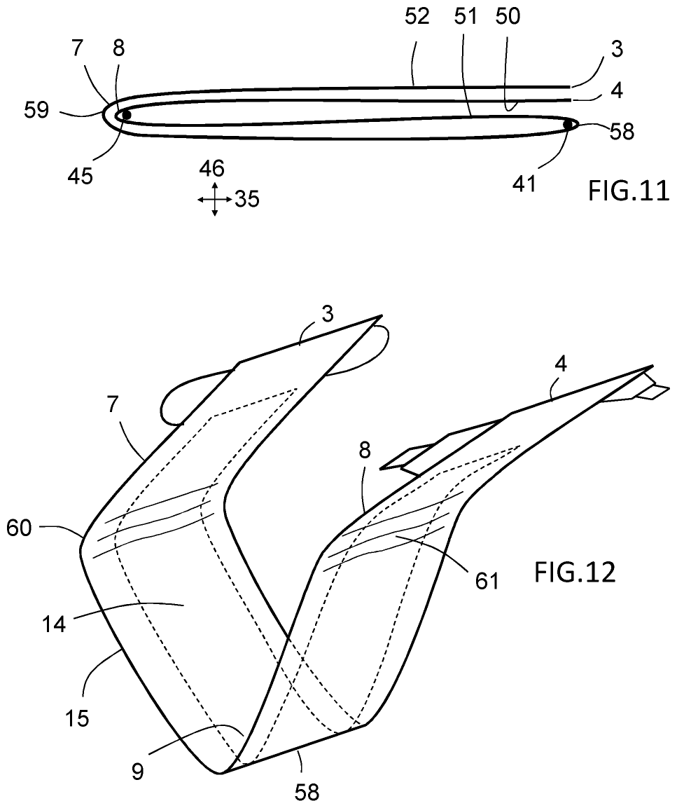

FIG. 11 illustrates schematically a side view of the quattro folded absorbent article of FIG. 10 having the outer front region 52 facing upwards in the figure, the first transverse fold line 41 located adjacent the folded edge 58 in the crotch portion 9 and the front waist edge 3 and back waist edge 4, the folded edge 59 located substantially coaxial with the second transverse fold line 45, and the outer surface 50 of the back portion 8 that is facing the outer surface 51 of the crotch portion 9. This folding is advantageous because the folding of the absorbent article 1 along second transverse fold line 45 results in a smaller radius fold of the back portion 8 and a larger radius fold of the front portion 7, as clearly illustrated in FIG. 11. Since the back portion 8 of the absorbent article typically has a smaller thickness than the front portion 7 the back portion can more easily be folded to have a smaller radius fold than the front portion without springing back to its unfolded state, and thus enabling forming an overall thinner folded absorbent article.

Moreover, a further advantage is improved pre-forming of the absorbent article 1 for better fit on the body of the user, as will be closed described below with reference to FIG. 12, which shows schematically a side view of the quattro folded absorbent article of FIG. 11 in a natural partly unfolded state.

A front fold 60 of the front portion 7 of the absorbent article 1 along the second transverse fold line 45 results in a desirable curve-forming effect of the absorbent article 1 because the front fold 60 assists a curved pre-forming with the topsheet 14 facing inwards, i.e. towards the radially inner side of the curve-shape. On the other hand, a back fold 61 of the back portion 8 of the absorbent article 1 along the second transverse fold line 45 results in an undesirable pre-forming effect of the back fold 61 because the resulting pre-forming is that the topsheet 14 faces outwards, i.e. towards the radially outer side of the curve at the back fold 61. However, the front fold 60 has a relatively strong desirable pre-forming effect on the article in the front 7, while the undesirable pre-forming effect of the back fold 61 is relatively weak due to lack or at least smaller amount of absorbent core 2 in the back portion 8. Consequently, the quattro folding while having the outer surface 50 of the back portion 8 facing the outer surface 51 of the crotch portion 9 results in improved pre-forming of the folded absorbent article.

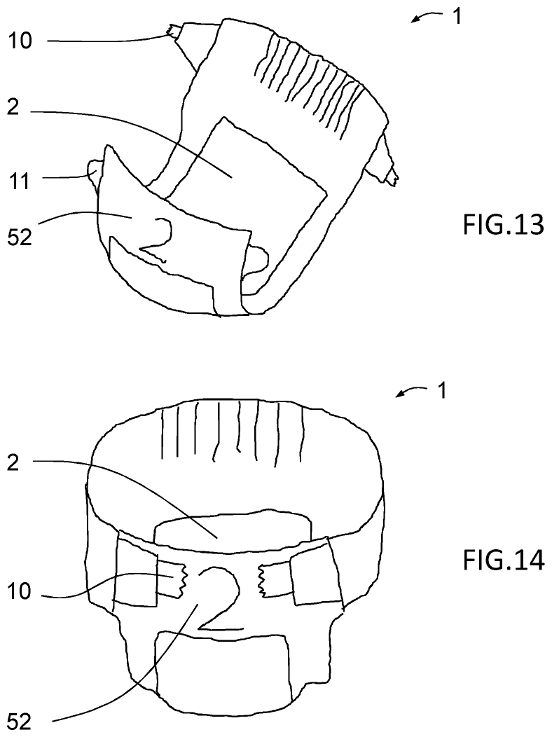

As schematically illustrated in FIGS. 13 and 14, the absorbent article may for example be an open baby diaper absorbent article 1, wherein FIG. 13 shows the article in an open state and FIG. 14 shows the article in a closed state, in which the fastening tabs are attached to a landing zone on the front portion 7.

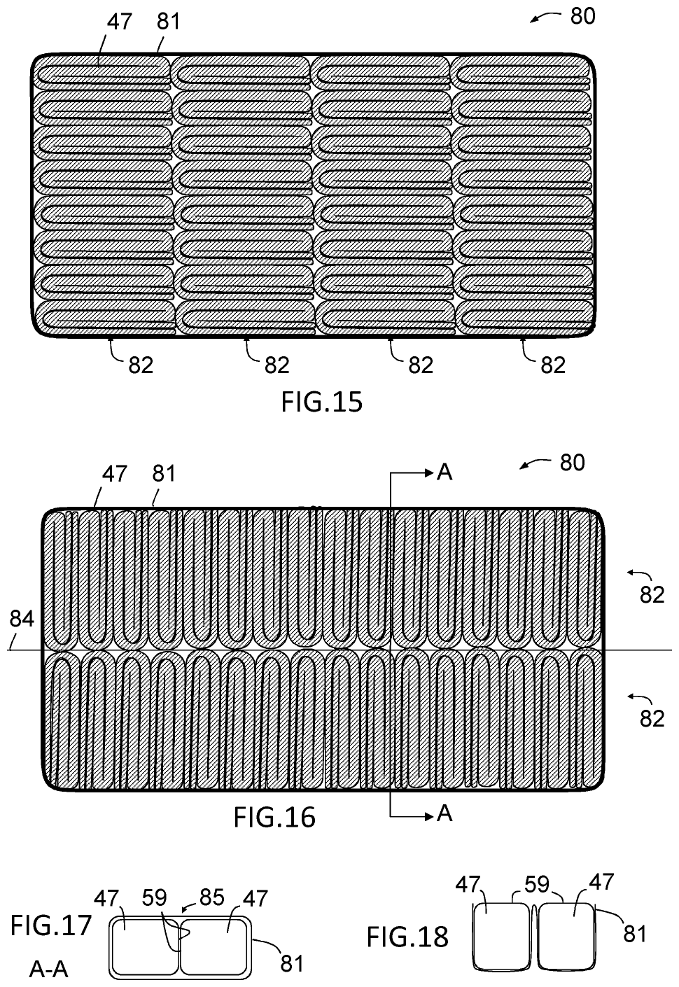

The disclosure further relates to a package 80 comprising a stack of folded absorbent articles 47 according to the disclosure above. FIGS. 15 and 16 illustrate schematically example packaging layouts of the folded absorbent articles 47 within a bag 81. FIG. 15 shows a package 80 that comprises four parallel stacks 82 of absorbent articles 1 located side-by-side. This packaging design enables by the relatively compact and small outer size of the quattro folded absorbent articles 47 increased stability and less amount of bag material per absorbent article, compared with a package with a single stack.

Alternatively, the quattro folded absorbent articles 47 may be packed in a package 80 that comprises two parallel stacks 82 of absorbent articles 1 located side-by-side. This type of packaging enables opening of the package 80 along a center line 84, for example by a weakening in the bag 81, and subsequently folding of the package along a fold line that is parallel with the center line 84.

For example, FIG. 17 schematically illustrates a cross-section along cut A-A in FIG. 16, wherein the quattro folded absorbent articles 47 of each stack 82 is arranged to have their folded edge 59 of the folded absorbent article 47 of FIG. 10 facing each other. A user may subsequently tear the bag 81 along a weakening 85 at center line 84 and thereafter fold the package such that two rows of folded absorbent articles 47 are visible and individual folded absorbent articles may be manually picked and removed from the package by grabbing the visible folded edge 59.

The disclosure further relates to a method for manufacturing a folded absorbent article 47, which absorbent article comprises, in an unfolded and planar state, a longitudinal center line, a transverse center line 30, a longitudinal length 32 extending from a front waist edge 3 to a back waist edge 4, and a transverse length 33 extending from a first side edge 5 to a second side edge 6, said absorbent article further comprising a front portion 7, a back portion 8 and a crotch portion 9, and fastening tabs 10 located on each transverse side of the back portion 8 for being releasably fastened to the front portion 7 when the absorbent article is in a fastened position The method comprises the steps of forming an absorbent article having an absorbent core sandwiched between a liquid-permeable topsheet and a liquid-impermeable backsheet and a flexure-resistance of less than 450.0 grams-force, specifically less than 300.0 grams-force, and more specifically less than about 230.0 grams-force and still more specifically less than 190.0 grams-force; folding each transverse side 37 of the absorbent article along respective longitudinal fold line 38, such that each of first and second transversely opposed side edges 5, 6 are folded onto a topsheet surface of the absorbent article thereby forming a firstly folded absorbent article 39; folding the firstly folded absorbent article 39 along a first transverse fold line 41 located in the region of the transverse center line 31 thereby forming a secondly folded absorbent article 42; and folding the secondly folded absorbent article 42 along a second transverse fold line 45 located approximately in the middle between the transverse center line 31 and a front waist edge 3 thereby forming a thirdly folded absorbent article 47.

Various types of materials may be used for the absorbent article 1. The topsheet 14 is arranged to face the wearer of the absorbent article 1 when worn. The topsheet 14 may be formed by a fluid permeable nonwoven fabric or film, which is made of thermoplastic synthetic fibres. The topsheet 14 may be sufficiently liquid-permeable to allow discharged body fluids to penetrate through the thickness of the topsheet 14. In addition, the topsheet 14 may be suitably manufactured from a material, which is compliant and soft feeling to the skin of the wearer. The topsheet 14 may consist of a single layer or have a laminate structure comprising a plurality of layers, for example, two or more layers. The layers may be made of the same material, or some or all the layers may be made of different materials.

The layer of the topsheet 14 or, for the case of a laminate structure, one, some, or all layers of the topsheet may be made of a single material or have plural portions made of different materials, e.g., within different parts of the wearer-facing surface of the topsheet.

The layer of the topsheet 14 or, for the case of a laminate structure, one, some or all layers of the topsheet may be a nonwoven material, a perforated plastic film, a plastic or textile mesh, or a liquid permeable foam layer.

The layer of the topsheet 14 or, for the case of a laminate structure, one, some, or all of the layers of the topsheet may be, for example, a hydrophilic, non-apertured nonwoven web of fibres, such as natural fibres, e.g., cotton or pulp fibres, synthetic fibres, e.g., polyester or polypropylene fibres, or a combination of these fibres.

The topsheet may have a basis weight in the range of 8-40 g/m². However, the disclosure is not limited to topsheets having this basis weight only.

Furthermore, the backsheet 15 may be constituted by a liquid-impermeable and breathable layer such as a polymeric film, for example a film of polyethylene or polypropylene. According to different embodiments, the materials, which may be used for the backsheet 15, include thin and flexible fluid impermeable plastic films, or fluid impermeable nonwoven materials, fluid impermeable foams and fluid impermeable laminates.

The backsheet 15 may be formed by a single layer, but may alternatively be formed by a multi-layered structure, i.e. a laminate, wherein at least one layer is fluid impermeable. Furthermore, the backsheet 15 may be elastic in any direction.

Furthermore, the backsheet 15 may have a laminate structure comprising a liquid barrier sheet and a nonwoven layer arranged on top of each other (not shown in detail in the drawings), wherein the nonwoven layer is arranged at an outer side away from the wearer of the absorbent article 1 when worn.

The nonwoven layer may be made of thermoplastic polymer material fibres or filaments. The nonwoven layer may be formed by a variety of different processes, such as spunbonding, air laying, meltblowing or bonded carded web formation processes. The nonwoven layer may be made of an SMS (spunbond/meltblown/spunbond) or SS (spunbond/spunbond) nonwoven material of polypropylene or bicomponent fibres of polypropylene and polyethylene, or of a combination of such materials. The nonwoven layer may have a basis weight in the range of 5-40 g/m².

The liquid barrier sheet may be made of a plastic material, for example a thermoplastic film material, and/or a nonwoven material. For example, the liquid barrier sheet may be formed as a plastic layer, e.g., a thermoplastic layer, or a plastic film, e.g., a thermoplastic film. Forming the liquid barrier sheet of a plastic material, such as a thermoplastic film material, allows for a particularly good printability of the liquid barrier sheet.

The liquid barrier sheet may be a liquid impermeable, breathable or non-breathable layer. The liquid barrier sheet may consist of a single layer or have a laminate structure with a plurality of layers, e.g., two or more layers, three or more layers, or four or more layers. The layers of the liquid barrier sheet may be laminated, bonded or attached to each other, for example, by thermos bonding and/or mechanical bonding, such as thermo-sealing, ultrasonic bonding, such as ultrasonic welding, an adhesive or adhesives, stitching or the like.

The liquid barrier sheet may be a breathable microporous film. The microporous film may be made of a material comprising at least two basic components, namely a thermoplastic elastomeric polyolefin polymer and a filler. These components and, in some embodiments, additional other components may be mixed together, heated and subsequently extruded into a mono-layer or multi-layer film using any one of various film-producing processes, such as cast embossed, chill and flat cast, and blown film processes.

Furthermore, the absorbent core 2 is provided between the topsheet 14 and the backsheet 15 to absorb the liquid, such as urine or other bodily fluids, which has passed through the topsheet 14. The absorbent core 2 may be made of one layer only, made from any suitable absorbent or liquid uptake material, such as one or more layers of cellulose fluff pulp, foam, fibre waddings or the like.

Each of the one or more absorbent layers of the absorbent core may have a homogeneous structure or a layered structure, i.e. an absorbent laminate of the same or different materials. Each of the one or more absorbent layers may have uniform or non-uniform thickness over the size of each respective absorbent layer. Similarly, the basis weight and composition may vary within the one or more absorbent layers. By way of example, an absorbent layer may comprise a mixture of absorbent and/or non-absorbent fibres and superabsorbent material, wherein the ratio of superabsorbent material to fibres may vary in the layer.

The absorbent core 2 may comprise suitable amounts of superabsorbent particles. Such superabsorbent material is well known in the field of absorbent articles, and is constituted by a water-swellable and water-insoluble material, which is capable of absorbing large quantities of fluid upon formation of a hydrogel. The absorbent core 2 may contain superabsorbent material in the form of fibres or particles of absorbent polymer material. For example, the superabsorbent material may be surface cross-linked, partially neutralized polyacrylates.

The superabsorbent material, e.g., the superabsorbent fibres or particles, may be mixed with other absorbent or liquid uptake material or materials, such as cellulose fluff pulp, and/or arranged in pockets or layers in the absorbent core 2. The amount of superabsorbent material and pulp in the absorbent core 2 may be 60-100% by weight superabsorbent material.

In other words, the absorbent core may comprise about 60-100 wt % SAP (Super Absorbent Polymer). If the absorbent core in a region of the absorbent core comprises less than 100 wt % SAP the remaining material may for example be primarily pulp material, or the remaining material may for example be only pulp material.

The absorbent core 2 may further comprise components for improving the properties of the absorbent core 2. For example, the absorbent core 2 may comprise a binder or binders, such as binder fibres.

Furthermore, as known by the skilled person, the various layers of the absorbent article 1 may be attached by adhesive material. Such adhesive is not shown in the drawings.

One or more additional layers may be provided in the absorbent article 1. For example, an acquisition layer may be arranged between the absorbent core 2 and the topsheet 14. Such an additional layer may for example be in the form of an airlaid layer, a spunlaced layer, a high-loft, foam or any other type of material layer, which may be used in an absorbent article to act as a liquid acquisition and absorption layer. The acquisition layer is adapted to quickly receive and temporarily store discharged liquid before it is absorbed by the absorbent core. Such acquisition layer may be composed of for example airlaid nonwoven, spunlaced nonwoven, high loft nonwoven or foam materials. An airlaid nonwoven may be produced with fluff, wood pulp, and here the fluff fibres are dispersed into a fast-moving air stream and condensed onto a moving screen by pressure and vacuum.

The absorbent core 2 may be wrapped in a core cover made of nonwoven or tissue material and be positioned between the topsheet 14 and the backsheet 15 during manufacturing of the absorbent article 1.

Furthermore, the absorbent core 2 and/or the topsheet 14 may comprise at least one additive material such as a skin care composition.

Although the disclosure has been described in relation to specific combinations of components, it should be readily appreciated that the components may be combined in other configurations as well which is clear for the skilled person when studying the present application. Thus, the above description of the example embodiments and the accompanying drawings are to be regarded as a non-limiting example of the disclosure and the scope of protection is defined by the appended claims. The disclosure may be varied from the examples provided while remaining within the scope of the claims. For example, the materials and dimensions used for the different layers forming the absorbent article 1 may be varied, as indicated above. The absorbent article may further include leg elastics, standing gathers, crotch and waist elastics, side panels, fastening systems etc. as known to the skilled man in the art and depending of the type of absorbent article intended.

What is claimed is:

1. A folded absorbent article comprising: an absorbent core sandwiched between a liquid-permeable topsheet and a liquid-impermeable backsheet, the absorbent article comprising, in an unfolded and planar state, a longitudinal center line, a transverse center line, a longitudinal length extending from a front waist edge to a back waist edge, and a transverse length extending from a first side edge to a second side edge, said absorbent article further comprising a front portion, a back portion and a crotch portion, and fastening tabs located on each transverse side of the back portion for being releasably fastened to the front portion when the absorbent article is in a fastened position, wherein the absorbent article has a flexure-resistance of less than 450.0 grams-force, wherein each transverse side of the absorbent article is folded along respective longitudinal fold line, such that each of first and second transversely opposed side edges are folded onto a topsheet surface of the absorbent article thereby forming a firstly folded absorbent article, wherein the firstly folded absorbent article is folded along a first transverse fold line located in a region of the transverse center line thereby forming a secondly folded absorbent article, and wherein the secondly folded absorbent article is folded along a second transverse fold line located approximately in the middle between the transverse center line and a waist edge thereby forming a thirdly folded absorbent article.

2. The folded absorbent article according to claim 1, wherein a thickness of the absorbent article in a region of the absorbent core is less than 7 mm.

3. The folded absorbent article according to claim 1, wherein a density of the absorbent article in a region of the absorbent core is higher than 0.16 g/cm³.

4. The folded absorbent article according to claim 1, wherein an inner surface of the back portion faces an inner surface of the front portion of the secondly folded absorbent article, and wherein an outer surface of the back portion faces an outer surface of the crotch portion of the thirdly folded absorbent article.

5. The folded absorbent article according to claim 1, wherein the absorbent core is constituted of one single core layer.

6. The folded absorbent article according to claim 1, wherein the absorbent core has a generally rectangular shape.

7. The folded absorbent article according to claim 1, wherein the absorbent article is an open baby diaper absorbent article.

8. The folded absorbent article according to claim 1, wherein the absorbent core comprises 60-100 wt % super absorbent polymer.

9. The folded absorbent article according to claim 1, wherein said absorbent core comprises at least one primarily longitudinally extending channel located at least in the crotch portion.

10. A package comprising: a stack of folded absorbent articles according to claim 1.

11. The package according to claim 10, wherein the package comprises at least two parallel stacks of folded absorbent articles located side-by-side.

12. A method for manufacturing a folded absorbent article, the absorbent article comprising, in an unfolded and planar state, a longitudinal center line, a transverse center line, a longitudinal length extending from a front waist edge to a back waist edge, and a transverse length extending from a first side edge to a second side edge, the absorbent article further comprising a front portion, a back portion and a crotch portion, and fastening tabs located on each transverse side of the back portion for being releasably fastened to the front portion when the absorbent article is in a fastened position, the method comprising:
forming an absorbent article having an absorbent core sandwiched between a liquid-permeable topsheet and a liquid-impermeable backsheet and a flexure-resistance of less than 450.0 grams-force;
folding each transverse side of the absorbent article along respective longitudinal fold line, such that each of first and second transversely opposed side edges are folded onto a topsheet surface of the absorbent article thereby forming a firstly folded absorbent article;
folding the firstly folded absorbent article along a first transverse fold line located in a region of the transverse center line thereby forming a secondly folded absorbent article; and
folding the secondly folded absorbent article located approximately in the middle between the transverse center line and a waist edge thereby forming a thirdly folded absorbent article.

13. The method according to claim 12, wherein the flexure-resistance of the absorbent article is less than 190.0 grams-force.

14. The folded absorbent article according to claim 1, wherein the flexure-resistance of the absorbent article is less than 190.0 grams-force.

15. The folded absorbent article according to claim 2, wherein a thickness of the absorbent article in a region of the absorbent core is less than 5 mm.

16. The folded absorbent article according to claim 3, wherein a density of the absorbent article in a region of the absorbent core is higher than 0.20 g/cm³.

17. The folded absorbent article according to claim 2, wherein a density of the absorbent article in a region of the absorbent core is higher than 0.16 g/cm³,
wherein an inner surface of the back portion faces an inner surface of the front portion of the secondly folded absorbent article, and wherein an outer surface of the back portion faces an outer surface of the crotch portion of the thirdly folded absorbent article,
wherein the absorbent core is constituted of one single core layer,
wherein the absorbent core has a generally rectangular shape,
wherein the absorbent article is an open baby diaper absorbent article,
wherein the absorbent core comprises 60-100 wt % super absorbent polymer, and
wherein said absorbent core comprises at least one primarily longitudinally extending channel located at least in the crotch portion.
